(19) United States
(12) Patent Application Publication
(10) Pub. No.: US 2024/0345012 A1
(43) Pub. Date: Oct. 17, 2024

(54) REUSABLE PORTABLE TESTING INSTRUMENT FOR TRANSDUCER

(71) Applicant: The Regents of the University of Colorado, a body corporate, Denver, CO (US)

(72) Inventors: Mujtaba Al Hubayl, Boulder, CO (US); Hayden Holtrop, Boulder, CO (US); Pandora Down, Boulder, CO (US); Brandon Muckenthaler, Boulder, CO (US); Jack McHale, Boulder, CO (US); Miguel Bermudez, Boulder, CO (US); Elliot Strand, Boulder, CO (US); Gregory Whiting, Boulder, CO (US); Luke Richard Borgerding, Boulder, CO (US)

(21) Appl. No.: 18/633,902

(22) Filed: Apr. 12, 2024

Related U.S. Application Data
(60) Provisional application No. 63/495,757, filed on Apr. 12, 2023.

(51) Int. Cl. G01N 27/04; G01N 33/487

(52) U.S. Cl. CPC G01N 27/04; G01N 33/487

(57) ABSTRACT

A testing apparatus includes a first section defining a recess to receive and align a transducer and an alignment protrusion. The testing apparatus further includes a second section hinge-ably coupled to the first section and defining a sample port and an alignment opening. The testing apparatus includes a gasket insert disposed within the sample port and defining at least one well configured to receive a fluid therein and align with a first portion of the transducer. A connector is coupled to the second section, the connector having a plurality of flexible or spring-loaded contact pins that align with a second portion of the transducer. The testing apparatus is movable between (i) an open configuration wherein the first section is disposed at an angle with respect to the second section and (ii) a closed configuration wherein the alignment protrusion is disposed within the alignment opening.

### CROSS-REFERENCE TO RELATED APPLICATIONS

This application claims priority to U.S. Provisional Patent Application No. 63/495,757, filed Apr. 12, 2023, which is incorporated herein by reference in its entirety.

### BACKGROUND

The global food crisis, exacerbated by factors such as population growth, soil degradation, and various environmental and political challenges, necessitates the development of technologies to enhance agricultural productivity and address the impending challenges. In response, efforts to improve plant growth and increase overall food yield have been begun. Hydroponic farming, in particular, presents advantages over traditional farming methods by utilizing less water and space while yielding higher food production. However, hydroponic farming services often come with significant costs and time requirements.

Recognizing this need for accessible solutions, printed sensors have been designed to measure specific elements and properties of hydropic fluid (c.g., nitrogen, potassium, and phosphorus concentrations). Such sensors enable frequent monitoring of nutrient levels in hydroponic fluid, yielding quick and actionable results. However, a comprehensive system or testing device for utilizing these sensors has not yet been developed.

Therefore, a need exists for reusable and efficient testing devices for utilizing and interacting with fluid testing sensors for hydroponic farming and other applications (c.g., biological testing).

### SUMMARY

Disclosed herein are systems, methods, and devices for providing accurate measurements of ion levels of selected elements from a sample fluid. For example, concentrations of nitrogen, potassium, and nitrogen may be measured in a hydroponic fluid sample. The disclosed testing apparatus integrates with screen-printed sensors (e.g., transducers with a printed portion that converts biological and/or chemical signals into analog signals) with the necessary electronics in a compact housing. To accomplish this, the housing encompasses portable electronic components, maintains uninterrupted electrical connections between the printed sensor and its designated electrical contact during sensor readings, and includes a sealed compartment for the hydroponic fluid to rest upon the active area of the transducer during measurements. The sensors were utilized to measure levels of the target element by applying specific voltages across the gate and source terminals and source and drain terminals while reading the current across the conductive source-to-drain channel.

The disclosed testing apparatus provides a portable and reusable system for quickly testing a sample fluid (e.g., in the field or on-site). The screen-printed sensors are biodegradable and, thus, disposable between uses. The disclosed testing apparatus provides a rapid changeover process for inserting a new sensor for a new testing operation.

In addition to hydroponic fluid sample testing (c.g., water, nutrients), the disclosed testing apparatus can be alternatively employed for biological testing, e.g., blood, saliva, or urine. In some embodiments, the testing can be employed for glucose concentration measurement or for the presence of chorionic gonadotropin (pregnancy tester), among other testers.

According to one aspect, a testing apparatus is disclosed, the testing apparatus including: a first section (e.g., a first component or a bottom shell) defining a recess configured to receive and align a transducer (e.g., biodegradable transducer or transducer device having a printed portion that converts biological and/or chemical signals into analog signals) and including an alignment protrusion extending from a first surface of the first section (e.g., inside surface of the bottom shell); a second section (e.g., a second component or a top shell) hingably coupled to the first section and defining (i) a sample port extending therethrough from a first surface (e.g., outside surface) of the second section to a second surface (e.g., inside surface) of the second section, and (ii) an alignment opening extending from the second surface of the second section towards the first surface of the second section, the alignment opening configured to accept the alignment protrusion; a gasket insert disposed within the sample port of the second section and defining at least one well configured to receive a fluid therein, wherein the at least one well is configured to align with a first portion of the transducer when placed in the recess of the first section; and a connector coupled to the second surface of the second section, the connector having a plurality of flexible or spring-loaded contact pins extending away from the second surface of the second section, the plurality of contact pins being alignable with a second portion of the transducer when placed in the recess of the first section, the second portion having electrodes contact spaced apart from, and electrically connected to, the first portion of the transducer, wherein the testing apparatus is movable between (i) an open configuration wherein the first surface of the first surface is spaced apart from and disposed at an angle with respect to the second surface of the second section and (ii) a closed configuration wherein the alignment protrusion of the first section is disposed within the alignment opening of the second section.

In some implementations, when the testing apparatus is in the closed configuration, the well is configured to receive a testing fluid, and the gasket insert forms a fluid-tight seal with the first portion of the transducer.

In some implementations, the testing apparatus includes a printed circuit board (c.g., a standard, rigid piece of electronics used to interrogate and/or read out data from the transducer) in electrical communication with the plurality of contact pins, the printed circuit board configured to selectively apply or measure an electrical signal (voltage and/or current) at each of the plurality of contact pins. In some implementations, the printed circuit board is configured to interrogate the contact pins by application of a voltage to a first electrode of the second portion transducer (e.g., to measure a concentration of a target element).

In some implementations, the printed circuit board is configured to interrogate the contact pins to measure a concentration of at least one target element selected from the group consisting of potassium, nitrogen, and phosphate. In some implementations, the printed circuit board is electrically coupled to the plurality of contact pins via a conductor routed through a hinge connecting the first section to the second section.

In some implementations, the transducer includes a potentiostat electrode (e.g., including three or more pins, c.g., a drain pin, a gate pin, and a source pin), amperostat electrodes, coulometry electrodes, or voltammetry electrodes.

In some implementations, the first section further defines an overflow well surrounding the recess configured to receive the transducer, wherein the overflow well collects and redirects excess fluid towards an overflow container in fluid communication with the overflow well. In some implementations, the well of the gasket insert includes three wells.

In some implementations, the alignment protrusion includes a tapered protrusion, and the alignment opening includes a tapered recess matching that of the alignment protrusion, wherein the tapered protrusion and tapered recess are configured to urge the transducer to an aligned position in the recess while the flattening the transducer as the testing apparatus moves from the open configuration to the closed configuration.

In some implementations, one of the first section or the second section includes a latch and the other one of the first section or the second section includes a latch receiver dimensioned to accept the latch, wherein in the closed configuration, the latch maintains the testing apparatus in the closed configuration.

In some implementations, the first section is configured to house the printed circuit board.

In some implementations, the plurality of contact pins are biased away from the second surface of the second section such that, in the closed configuration, the plurality of contact pins provide a spring force against the second portion of the transducer.

In some implementations, the recess of the first section further includes a notch configured to align and orient the transducer and a divot configured to facilitate the removal of the transducer.

According to another aspect, a method of testing a sample fluid using the testing apparatus is disclosed.

In some implementations, the sample fluid includes a medical sample (e.g., blood, saliva, urine, etc.) In some implementations, the sample fluid includes an agricultural or hydroponic sample (e.g., hydroponic fluid, plant sap, processed soil into liquid form, etc.) In some implementations, the sample fluid includes an environmental sample (e.g., a sample extracted from rivers, lakes, wastewater, sewage, ocean, etc.) In some implementations, the sample fluid includes a chemical manufacturing sample (e.g., oil, pharmaceuticals, etc.)

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIG. 1A shows a system diagram of the disclosed testing apparatus, including the mechanical and electrical components thereof, according to one implementation.

FIG. 1B shows a system diagram of a testing apparatus, according to another implementation.

FIGS. 2A-2C show the testing apparatus of FIG. 1, according to one implementation.

FIGS. 3A-3C show the transducer and related diagrams, according to various implementations.

FIG. 4A shows a detailed view of the first section of the testing apparatus, according to one implementation.

FIG. 4B shows a detailed view of the connector, including the plurality of contact pins, according to one implementation.

FIGS. 4C and 4D provide detailed views of the overflow mechanism of the testing apparatus, according to one implementation.

FIGS. 4E and 4F show details on the gasket insert, according to one implementation.

FIG. 4G shows details on the latch of the testing apparatus, according to one implementation.

FIG. 4H shows the printed circuit board of the testing apparatus, according to one implementation.

FIG. 4I shows a top view and detailed view of the transducer with exemplary dimensions in millimeters, according to one implementation.

FIGS. 4J and 4K show example circuits for a potentiostat and a voltammetry circuit, respectively.

FIG. 5 depicts a flowchart of a method of use of the testing apparatuses disclosed herein, according to one implementation.

FIG. 6A shows a schematic for connecting breakout boards of this disclosure, according to one implementation.

FIG. 6B shows a board layout for connecting breakout boards of this disclosure, according to one implementation.

FIG. 6C shows a schematic for connecting a female socket receptor to a 45-degree pin header, according to one implementation.

FIG. 7A shows the connection pin test setup including the connection pins placed within a compression testing block on a universal testing machine (UTM), according to one implementation.

FIG. 7B shows a plot of the four trials showing the curves for force vs displacement, according to one implementation.

FIG. 8A shows the compression testing blocks used on a universal testing machine (UTM) with the gasket testing device therebetween, according to one implementation.

FIG. 8B shows a plot of force vs displacement for the gasket sealing test, according to one implementation.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, a reusable portable testing instrument for transducers (e.g., biodegradable transducer) is shown, according to various implementations.

### System Diagram

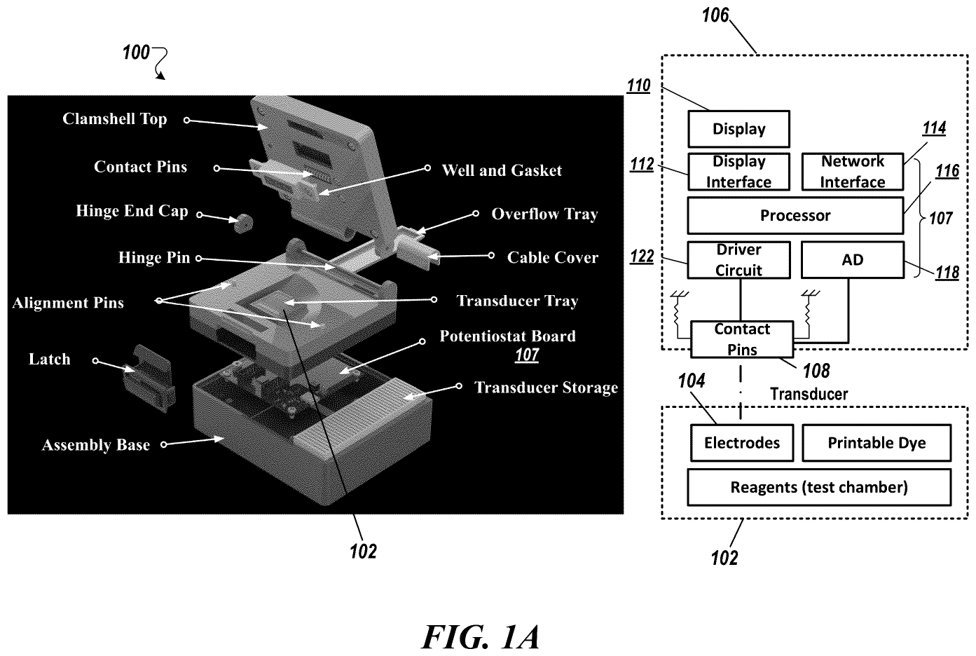

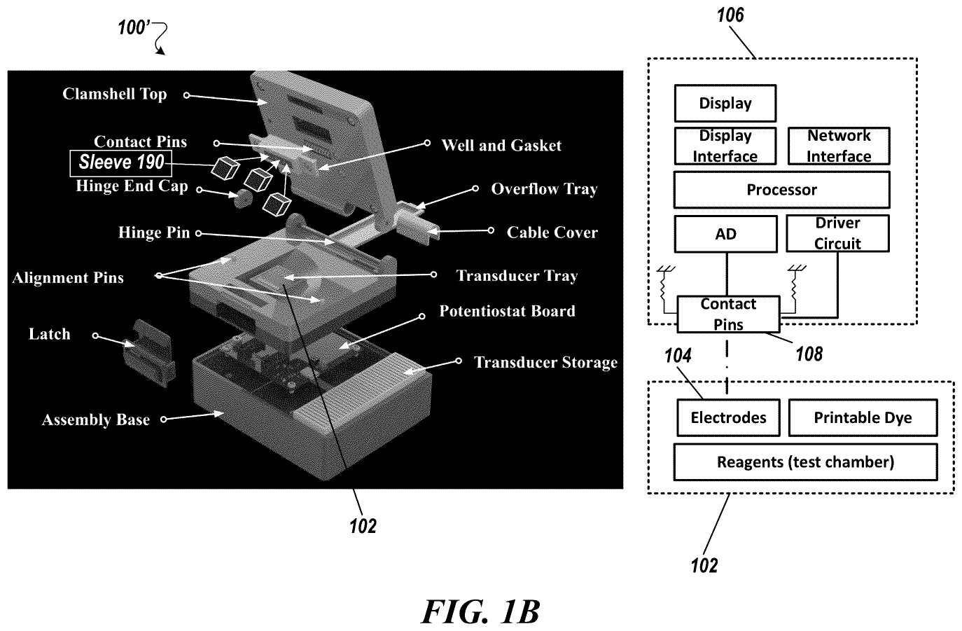

FIG. 1A shows a system diagram of the disclosed testing apparatus, including the mechanical and electrical components thereof. The testing apparatus 100 is used for hydroponic testing wherein a sample of hydroponic fluid is deposited into the wells of the gasket insert. However, in other implementations, the testing apparatus may be used for a different fluid and/or testing operation.

As shown, the testing apparatus 100 includes a transducer 102 (e.g., biodegradable transducer with a printed portion that converts biological and/or chemical signals into analog signals) that includes electrodes 104 (e.g., the printed portion). In some implementations, the transducer 102 comprises a potentiostat electrode comprising three or more pins (c.g., a drain pin, a gate pin, and a source pin).

The testing apparatus 100 further includes a controller 106 (e.g., a single board computer, e.g., shown as “PCB” 106). The controller 106 includes a connector mounted to the inner surface of the housing of the apparatus, the connector having a plurality of flexible or spring-loaded contact pins 108 to form an electrical connection with the electrodes 104 of the transducer 102. The controller 106 may include controller board 107 (shown as “Potentiostat Board” 107) that may include a display interface 112, network interface 114, processor 116, AD 118, and driver circuits 122. The controller 106 may additionally include a display 110 (not shown).

A controller 106, as a potentiostat, includes electronic hardware to control a three electrode cell to run an electroanalytical experiment. The controller 106 can maintain the potential of a working electrode, via a voltage source and operational amplifier 122, at a constant level with respect to a reference electrode by adjusting the current at an auxiliary electrode. The measured current can be sensed by an analog-to-digital conversion circuit 118 to provide a measurement of the sample.

The transducer 102 can be a biodegradable transducer. In some implementations, the transducer includes a flexible base substrate/material (e.g., paper-based) with a transducer printed thereon (e.g., wherein the “transducer” is a printed portion on the base substrate that converts biological and/or chemical signals into analog signals. In some embodiments, transducer is a paper-based transducer having a flexible paper-based material with conductive dye formed hereon.

While shown with a potentiostat, the controller 106 may be configured with other circuitries, such as, but not limited to amperostat, coulometry, and voltammetry, among others. An amperostat (also referred to as a galvanostat) is configured to keep the current through an electrolytic cell in coulometric titrations constant, disregarding changes in the load itself. Potentiometry circuit can measure difference in electrode potentials, coulometry measures a cell's current over time), and voltammetry measures a cell's current while actively altering the cell's potential).

FIG. 1B shows a system diagram of a testing apparatus 100′ that is substantially similar to the testing apparatus 100 of FIG. 1A. However, the testing apparatus 100′ is used for biological testing wherein a biological sample is deposited into a sleeve 190 inset into the well of the gasket insert. In this way, the biological sample does not contaminate other portions of the testing apparatus 100′.

### Testing Apparatus

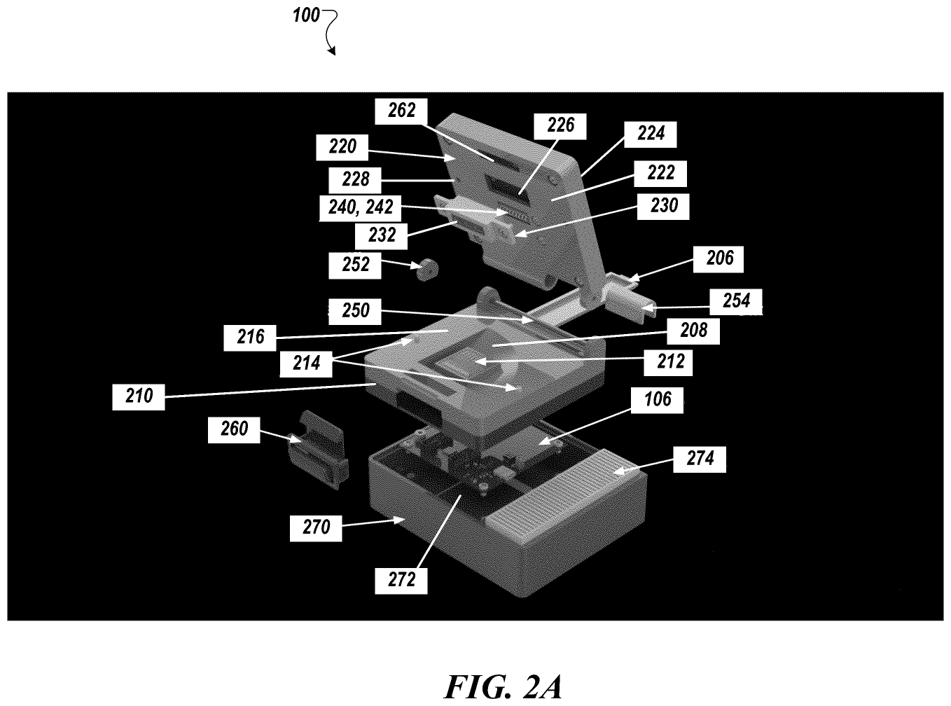

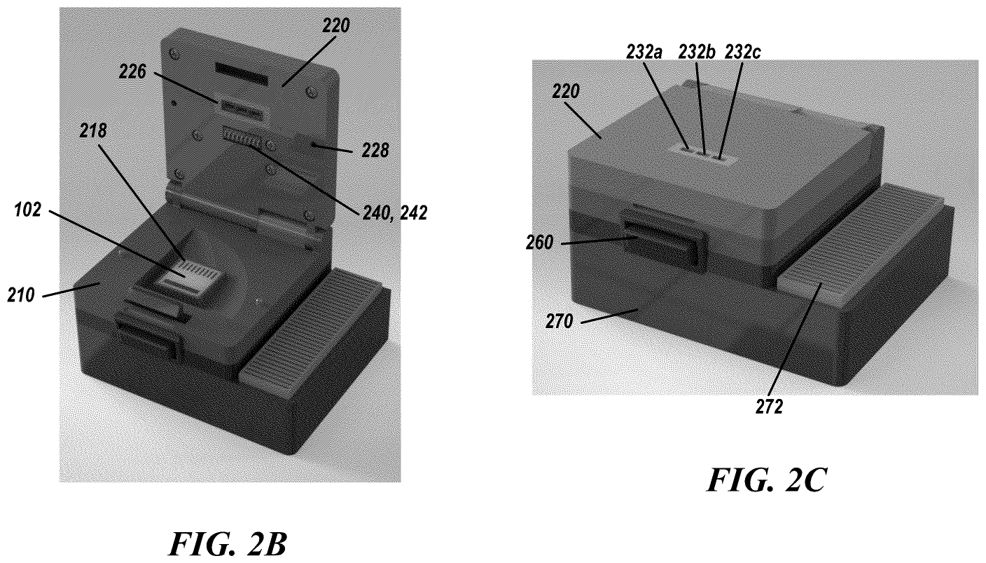

FIGS. 2A-2C show the testing apparatus 100 of FIG. 1, according to one implementation. Specifically, FIG. 2A shows an exploded view, FIG. 2B shows an open configuration, and FIG. 2C shows a closed configuration of the testing apparatus 100. The testing apparatus 100 includes, among other elements, a first section 210, a second section 220, a gasket insert 230, a connector 240, and the printed circuit board 106.

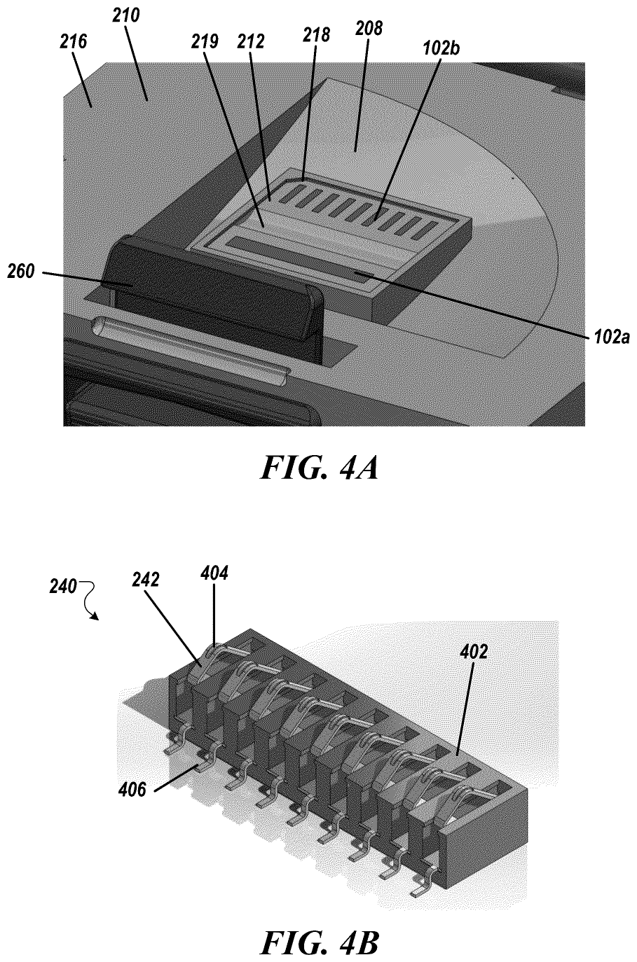

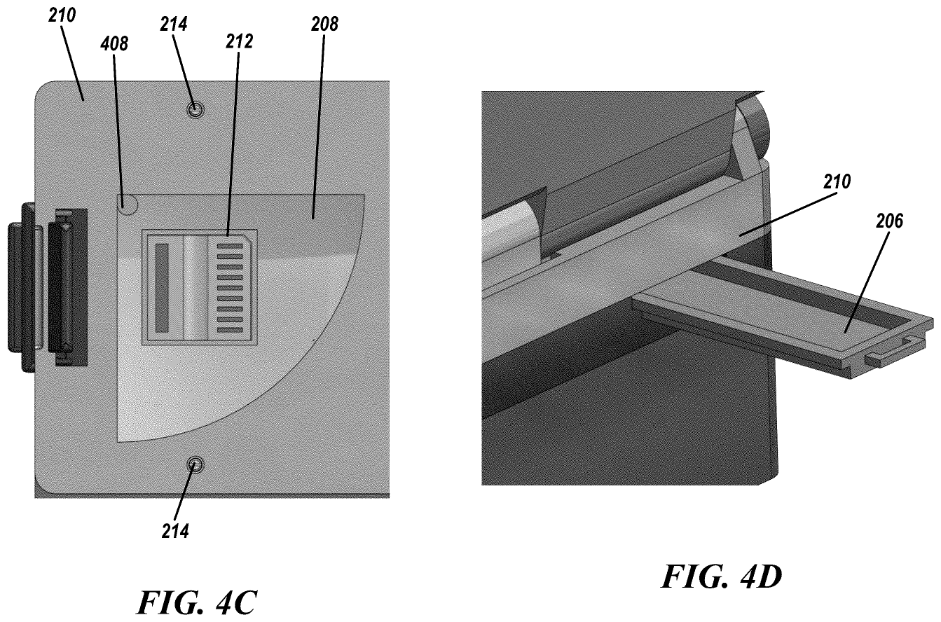

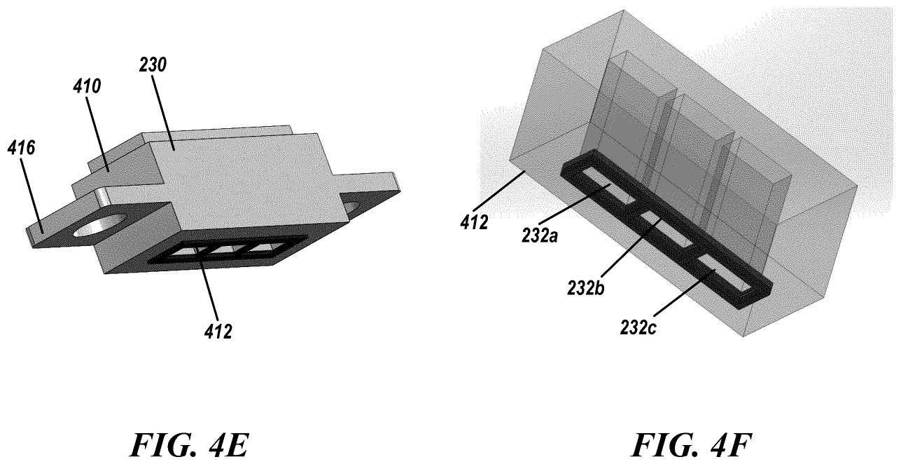

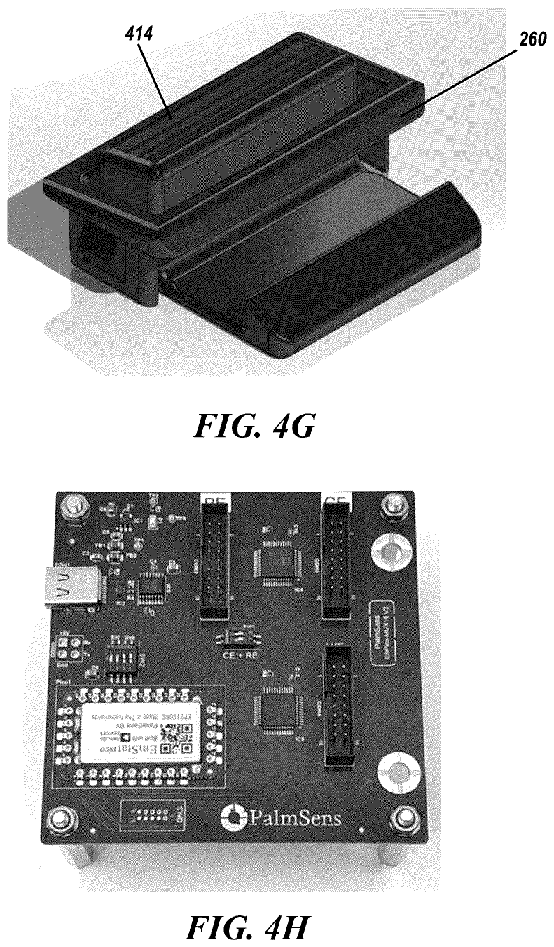

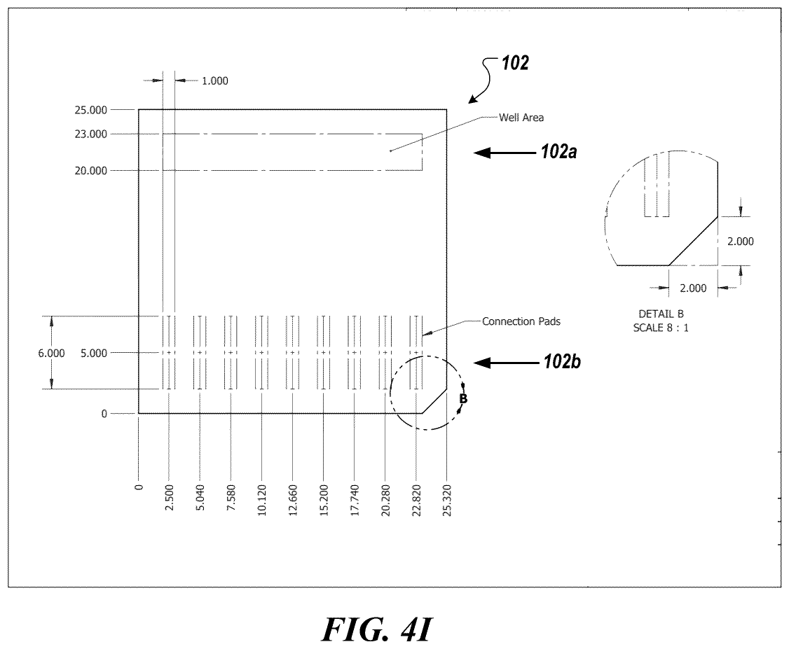

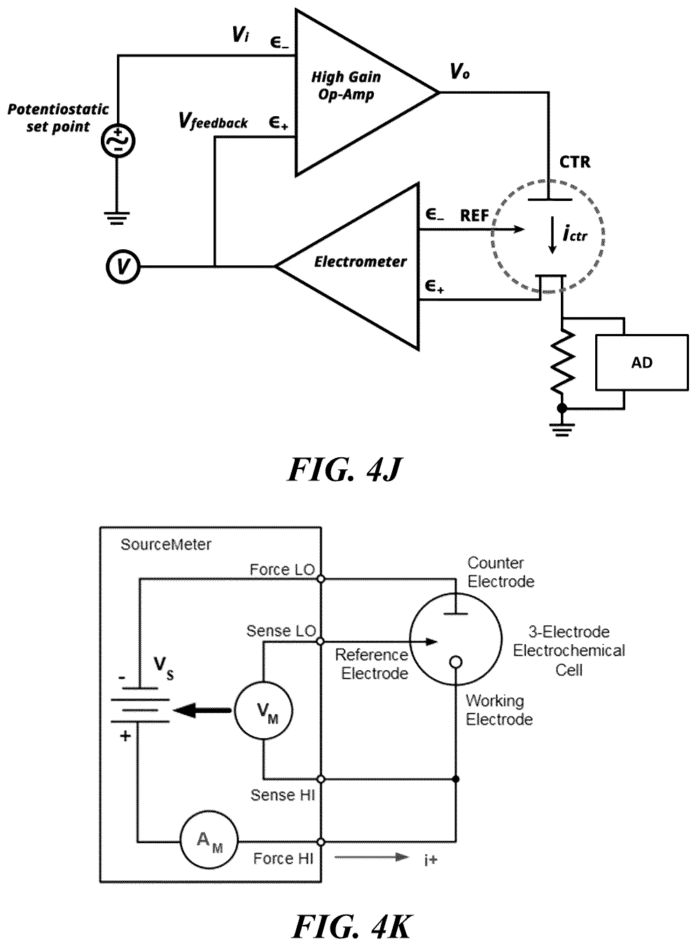

The first section 210 (e.g., a first component or a bottom shell of the testing apparatus) defines a recess 212 configured to receive and align the transducer 102. The recess 212 of the first section 210 includes a notch 218 configured to align and orient the transducer 102 and a divot 219 configured to facilitate removal of the transducer 102 (e.g., as shown in more detail in FIG. 4A). The first section 210 further includes an alignment protrusion 214 extending from a first surface 216 of the first section 210 (e.g., an inside surface of the bottom shell). As shown, the first section 210 includes two alignment protrusions 214—one on either side of the testing apparatus 100.

The first section 210 further defines an overflow well 208 surrounding the recess 212. The overflow well 208 collects and redirects excess fluid towards an overflow container 206 in fluid communication with the overflow well 208.

The first section 210 sits within and/or on top of an assembly base 270. The assembly base 270 defines a central cavity 272 housing the printed circuit board 106. However, in other implementations, the printed circuit board is disposed within the first section. The assembly base 270 further includes a transducer storage array 274 configured to house a plurality of transducers 102 or transducer devices.

The second section 220 (e.g., a second component or a top shell of the testing apparatus, shown as “clamshell top” in FIG. 1A) is hingably coupled to the first section 210. Specifically, a hinge pin 250 on the first section 210 connects to the second section 220, along with a hinge end cap 252. An optional cable cover 254 is disposed over the hinge pin 250 to facilitate cable routing as further described below. The hinge mechanism allows the second section 220 to freely rotate with respect to the first section 210. In some implementations, a constant torque hinge is used so that the second section remains at a fixed angle with respect to the first section when released.

The testing apparatus 100 is movable between (i) an open configuration wherein the first surface 216 of the first section 210 is spaced apart from and disposed at an angle with respect to the second surface 224 of the second section 220 (e.g., as shown in FIG. 2B), and (ii) a closed configuration wherein the alignment protrusion 214 of the first section 210 is disposed within the alignment opening 228 of the second section 220 (e.g., as shown in FIG. 2C).

A latch 260 is coupled to the first section 210, and a latch receiver 262 is defined in the second section 220. However, in other implementations, the latch may be coupled to the second section with the latch receiver in the first section. The latch receiver 262 is dimensioned to accept the latch 260, wherein in the closed configuration the latch 260 maintains the testing apparatus 100 in the closed configuration.

The second section 220 includes a first surface 222 (e.g., an outside surface) and a second surface 224 (e.g., an inside surface) opposite and spaced apart from the first surface 222. The second section 220 defines a sample port 226 extending through the second section 220 from the first surface 222 to the second surface 224.

The second section 220 further includes an alignment opening 228 extending from the second surface 224 of the second section 220 towards the first surface 222 of the second section 220. The alignment opening 228 is configured to accept the alignment protrusion 214 of the first section 210. In some implementations, the alignment protrusion includes a tapered protrusion and the alignment opening comprises a tapered recess matching that of the alignment protrusion. In some implementations, the tapered protrusion and tapered recess are configured to urge the transducer to an aligned position in the recess while the flattening the transducer as the testing apparatus moves from the open configuration to the closed configuration. In some implementations, the first section includes an alignment opening while the second section includes a corresponding alignment protrusion.

In some implementations, the testing apparatus, including the first section and the second section, are 3D printed components. In some implementations, the testing apparatus comprises plastic material formed from a Fused Deposition Modeling (FDM) process or a Selective Laser Sintering (SLS) process. In some implementations, the testing apparatus includes heat set inserts providing a threaded connection point for a fastener.

The gasket insert 230 is disposed within the sample port 226 of the second section 220. The gasket insert 230 defines at least one well 232-shown as three wells 232a, 232b, and 232c. The wells 232 are configured to receive a fluid therein. The wells 232 are also configured to align with a first portion 102a of the transducer 102 (e.g., a fluid testing portion) when the transducer 102 is placed in the recess 212 of the first section 210. When in the closed configuration, the wells 232 are configured to receive a testing fluid, and the gasket insert 230 forms a fluid-tight seal with the first portion 102a of the transducer 102.

The connector 240 is coupled to the second surface 224 of the second section 220. The connector 240 includes a plurality of flexible or spring-loaded contact pins 242 extending away from the second surface 224 of the second section 220. The plurality of contact pins 242 are alignable with a second portion 102b of the transducer 102 (e.g., an electrical coupling portion opposite from the fluid testing portion) when the transducer 102 is placed in the recess 212 of the first section 210. The plurality of contact pins 242 are biased away from the second surface 224 of the second section 220 such that, in the closed configuration, the plurality of contact pins 242 provide a spring force against the second portion 102b of the transducer 102. The second portion 102b of the transducer 102 includes electrodes 104 spaced apart from, and electrically connected to, the first portion 102a of the transducer 102.

The printed circuit board 106 is disposed in the central cavity 272 of the assembly base 270. The printed circuit board 106 is in electrical communication with the plurality of contact pins 242 (e.g., via a conductor routed between the first and second sections via the hinge). The printed circuit board 106 is configured to selectively apply or measure an electrical signal (voltage or current) at each of the plurality of contact pins 242. Furthermore, the printed circuit board 106 is configured to interrogate the plurality of contact pins 242 by application of a voltage to a first electrode of the second portion 102b of the transducer 102 (e.g., to measure a concentration of a target element). In some implementations, the printed circuit board is configured to measure a concentration of at least one target element selected from the group consisting of potassium, nitrogen, and phosphate. In some implementations, the printed circuit board is a standard (e.g., off-the-shelf) component including a rigid piece of electronics used to interrogate and/or read out data from the transducer.

In use, a transducer 102 is aligned and placed within the recess 212 of the first section 210. The testing apparatus 100 is then moved to the closed configuration with the latch 260 holding the first section 210 and the second section 220 against each other. The gasket insert 230 contacts the first portion 102a of the transducer 102 to form a fluid-tight seal. At the same time, the plurality of contact pins 242 contact the second portion 102b of the transducer 102 to exert a spring biasing force and maintain electrical contact.

Then, a sample fluid is deposited into the wells 232 defined by the gasket insert 230 such that the sample fluid contacts the first portion 102a of the transducer 102. The sample fluid may include an agricultural or hydroponic sample (e.g., hydroponic fluid, plant sap, processed soil into liquid form, etc.). In other implementations, the sample fluid is a medical sample (e.g., blood, saliva, urine, etc.). In other implementations, the sample fluid is an environmental sample (e.g., a sample extracted from rivers, lakes, wastewater, sewage, ocean, etc.). In other implementations, the sample fluid is a chemical manufacturing sample (c.g., oil, pharmaceuticals, etc.).

The printed circuit board 106 sends signals to the plurality of contact pins 242 to selectively apply or measure an electrical signal (voltage or current) at each of the plurality of contact pins 242 corresponding to the second portion 102b of the transducer 102. The measured change in current corresponds to a measured concentration of an element in the sample fluid (c.g., a concentration of potassium, nitrogen, or phosphorus).

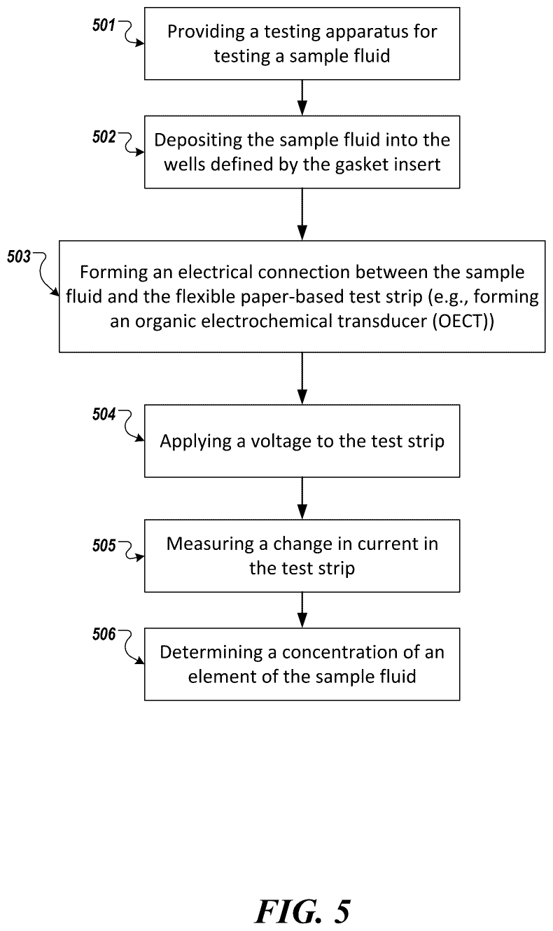

Once a test is complete and the concentration of elements in the sample fluid is collected, the testing apparatus 100 is opened, and the transducer 102 removed. Another transducer 102 may be placed in the recess 212 for additional tests (e.g., see FIG. 5 and the method flowchart therein).

### Example Transducer

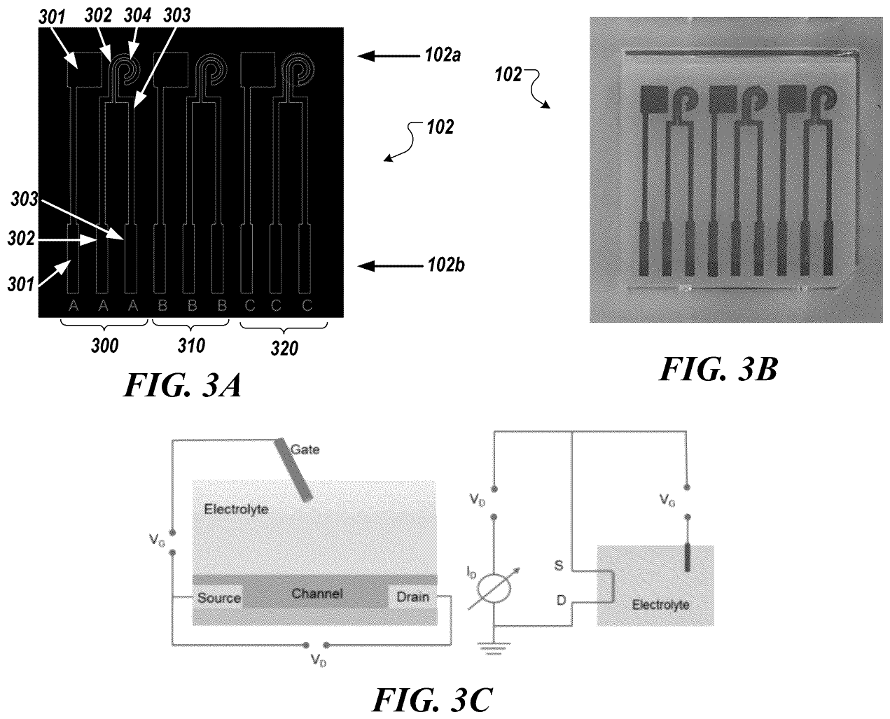

FIGS. 3A-3C show details of the transducer 102, according to one implementation. Specifically, FIG. 3A shows the layout of a transducer, FIG. 3B shows an image of a physical transducer formed from a die-cutting process, and FIG. 3C shows electronic diagrams describing the function and operation of the transducer.

The layout of the transducer 102 includes three sets of terminals forming three transducers or sensors (e.g., the printed portion) labeled A, B, and C. These three transducers (printed portions) correspond to the three elements measured (e.g., nitrogen, potassium, and phosphorus). Each transducer/sensor includes a gate, a source, and a drain terminal, arranged left to right in FIG. 3A. For example, the first transducer 300 includes a gate 301, a source 302, and a drain 303. The second transducer 310 and the third transducer 320 include a similar set of terminals.

The first portion 102a of the transducer 102 (where the testing fluid will contact the transducer 102) is shown with the changing geometries of the electrodes. The second portion 102b of the transducer 102 (where the plurality of contact pins 242 will contact the transducer 102) is shown on the opposite side with uniform contact points.

The first transducer 300 is an organic electrochemical transducer (OECT) which operates by applying a voltage at the gate 301, inducing the movement of charged ions into or out of a conductive polymer channel 304 positioned between the source 302 and the drain 303. The process leads to a modification in the channel's 304 resistivity. By applying a voltage across this channel 304, the resulting current varies in accordance with the concentration of the charged ions present in the hydroponic solution. This mechanism enables the sensor to exhibit sensitive and selective detection of nitrogen, potassium, and phosphorus, which are vital elements in diverse environmental and agricultural applications. The potentiostat will function to hold the gate to source voltage (Vgs) and the drain to source voltage (Vds) constant, while the current from the source terminal is measured and correlated with the ion concentrations.

FIG. 3C shows a layout of the electrical diagram formed between the drain, source, gate, and sample fluid (shown as “electrolyte”).

### Mechanical Sub-Components

FIG. 4A shows a detailed view of the first section 210 including the overflow well 208 and the recess 212. FIG. 4A shows the notch 218 and the divot 219 of the recess 212. The notch 218 is configured to align the transducer 102, having a corresponding notch on one corner, in the recess 212. Furthermore, the divot 219 facilitates removal of the transducer 102 from the recess 212.

FIG. 4B shows a detailed view of the connector 240 including the plurality of contact pins 242 disposed within a housing 402. Each of the plurality of contact pins 242 includes a contact point 404 at the apex of the plurality of contact pins 242. As the contact point 404 contacts the corresponding electrode on the second portion 102b of the transducer 102, the plurality of contact pins 242 compress into the housing 402. A resulting spring force is applied from the plurality of contact pins 242 to the transducer 102. While nine pins are provided in the plurality of contact pins 242 includes nine individual pins corresponding to the three sensors having three electrodes each, in other implementations a different number of pins is provided. For example, in other implementations, 1, 2, 3, 4, 5, 6, 7, 8, 10, 12, 15, 18, 21, 24, 27, or 30 pins may be used.

The connector 240 also includes secondary contacts 406 extending from the side of the housing 402. The secondary contacts 406 are configured to connect to the printed circuit board (e.g., via a ribbon cable).

FIGS. 4C and 4D provide detailed views of the overflow mechanism of the testing apparatus 100. For example, the top view of the first section 210 in FIG. 4C shows the overflow well 208 with an opening 408 at the bottom of the overflow well 208. Any excess fluid in the overflow well 208 flows into the overflow container 206, which is slidable out of, and back into, the first section 210. For example, when the second section 220 is opened following a testing operation, excess sample fluid may flow into the overflow well 208 and into the overflow container 206, which acts as a removable collection tray.

FIGS. 4E and 4F show details on the gasket insert 230. Specifically, FIG. 4E shows the gasket insert 230 unattached to the testing apparatus 100, and FIG. 4F shows a transparent view of the gasket insert 230. The gasket insert 230 includes a gasket housing 410 with a flexible insert 412. The gasket insert 230 includes flanges 416 on either side of the gasket insert 230 configured to accept a fastener and secure the gasket insert 230 to the second section 220. The flexible insert 412 defines the three wells 232a, 232b, and 232c therein. The flexible insert 412 comprises a flexible material (e.g., rubber) configured to compress when the testing apparatus 100 is moved to the closed configuration. The compressed flexible insert 412 forms the fluid tight seal with the transducer 102.

FIG. 4G shows the latch 260 of the testing apparatus 100. The latch 260 shown is the Southco 80-202 and has a maximum recommended force loading of 90 N (˜20 lbs) with a 100,000-cycle lifetime. Additionally, the latch 260 comes with the added convenience of being a “Push-to-Close” actuation, such that simply closing the top lid (e.g., the second section) allows the lid to latch without any complicated secondary locking mechanism. Conversely, pressing the button 414 on the side of the latch 260 will release the latch 260, allowing the device to be opened and the transducer to be retrieved.

FIG. 4H shows the printed circuit board 106, according to one implementation. The circuit board shown is an EmStat Pico MUX16 board. The board in FIG. 4H uses a Palmsens EmStat Pico Potentiostat which is connected via ribbon cable (e.g., the conductor) to the plurality of contact pins 242. In some implementations, the potentiostat is placed on standoffs in the base of the device (e.g., the assembly base 270) allowing for the device to exist as a single self-contained unit requiring only a single cable to interface with any computer. In some implementations, a USB-C port connected to the board is disposed in a side of the assembly base 270.

FIG. 4I shows a top view and detail view of the transducer 102 with exemplary

dimensions in millimeters. The transducer 102 in FIG. 41 is a 25.32 mm×25 mm×100 nm printed circuit with alignment notch. However, in other implementations, the transducer has dimensions matching that of the testing apparatus wherein the testing apparatus is a handheld or benchtop device. The transducer 102 shown has dimensions substantially matching the recess 212 of the first section 210 of the testing apparatus 100. The nine contact pads on the transducer 102 are shown on the second portion 102b, and the well area is shown on the opposite first portion 102a.

FIGS. 4J and 4K show example circuits for a potentiostat and a voltammetry circuit, respectively.

FIG. 5 depicts a flowchart of a method of use of the testing apparatuses disclosed herein. Step 501 includes providing a testing apparatus for testing a sample fluid. This could include the testing apparatus 100 of FIG. 1A and 2A-2C, or a different testing apparatus for a different testing fluid (e.g., the biological sample testing device of FIG. 1B). Step 502 includes depositing the sample fluid into the wells defined by the gasket insert. Again, the sample fluid could be any fluid for testing disclosed herein.

Step 503 includes forming an electrical connection between the sample fluid and the transducer (e.g., forming an organic electrochemical transducer (OECT)). This step occurs when the sample fluid forms a connection between the gate, source, and drain electrodes of the transducer, as shown in the example in FIG. 3C.

Step 504 includes applying a voltage to the transducer (e.g., applying a voltage at the gate). Step 505 includes measuring a change in current in the transducer (e.g., measuring a change in current at the drain and/or the equivalent resistance between the source and the drain). Step 506 includes determining a concentration of an element of the sample fluid (e.g., phosphorus, nitrogen, and/or potassium). The concentration is based on the changed current and/or resistivity measured across electrodes of the transducer.

### Experimental Results and Additional Examples

### Breakout Boards

Two breakout boards were utilized to establish a connection between the EmStat MUX16 board and the sensor terminals through the pin connections. This arrangement enabled the utilization of a single nine-wire ribbon cable, which can conveniently traverse the hinge connecting the upper and lower electronic hardware components.

To facilitate the connection of the surface-mounted deflection pins with a pin header, a compact breakout board was used. This breakout board will enable the attachment of the deflection pins to a pin header, which can then be connected to a ribbon cable. By employing this configuration, the ribbon cable can be routed through the housing hinge to reach the potentiostat located below.

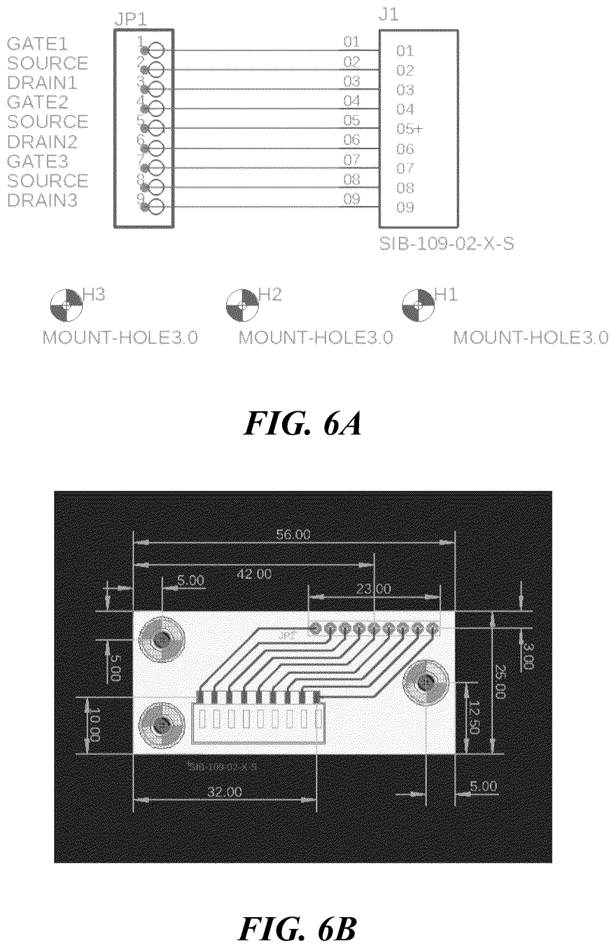

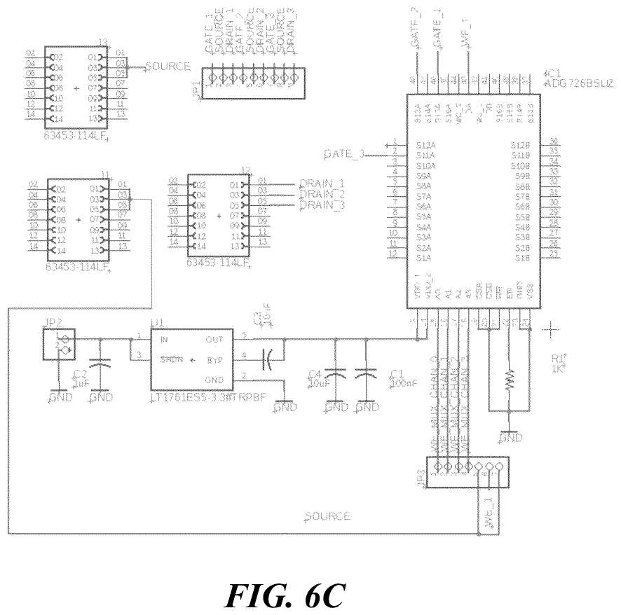

To implement this design, a small printed circuit board (PCB) was developed. The PCB featured surface-mount deflection pins located on the bottom side to establish contact with the sensor pads. On the top side, a 45-degree pin header with a 2.54 mm pitch was mounted. Since the pin header was through-hole mounted, the traces on the PCB could remain on the bottom side. However, in other implementations, the printed circuit board (PCB) may be a PCB not unique to this disclosure but instead any rigid piece of electronics used to interrogate and/or read out data from the transducer. FIG. 6A shows the schematic for connecting the 45-degree pin head (on the left of the figure, 9 pos, 2.54 mm pitch) to a Samtex Inc. Spring Compression Contact connector (on the right of the figure, 9 pos, 2.54 mm pitch), according to the experimental implementation.

The board layout is shown in FIG. 6B, in which the pin header on the right is offset from the center by 2 cm to allow the crimp-connected ribbon to align with the slot in the hinge.

An additional custom breakout board was employed to establish an interface with the three male pin headers on the EmStat MUX16 board, namely CON2, CON3, and CON4. These pin headers correspond to the multiplexor channels of channel 0 working electrode (WE0), counter electrode (CE0), and reference electrode (RE0). Female socket receptors matching the pitch and position were utilized so that the breakout board may be directly plugged into CON2, CON3, and CON4. FIG. 6C shows the schematic for connecting the female socket receptor (interfacing with male pin headers on the MUX16 board) to the 45-degree pin header (9 pos, 2.54 mm pitch).

Given that the EmStat MUX16 board only multiplexes a single channel of the potentiostat, this breakout board assumed the responsibility of multiplexing the second channel. In some embodiments, the board was connected to the power (5Vcc) and ground of the EmStat MUX16 board. By doing so, an additional multiplexor (ADG726BSUZ) could be employed on this board for work electrode one (WE1). As the power drawn from CON5 on the MUX16 board amounts to 5V, a voltage regulator (LT1761ES5-3.3 #PBF) was employed to reduce it to 3.3V, which was then utilized to power the multiplexor.

Furthermore, WEI, CEI, and REI were connected to the upper board alongside the four digital channels addressing the WE0 multiplexor located on the bottom board (WE_MUX_CHAN_0-WE_MUX_CHAN_3). These channels were selectively set to either HIGH or LOW outputs in various combinations to designate the output channel of the multiplexor. By aligning the new multiplexor with the same addressing pins as the WEO multiplexor, both multiplexors transmit their output signals to the corresponding terminal on their respective chips. Consequently, if the WEO multiplexor is currently outputting to SA11, the WEI multiplexor will simultaneously output to SA11.

To establish connections between these channels and the upper board, wires were directly soldered onto the potentiostat terminals for these channels and were routed to the JP3 pin header associated with these channels. From there, WEI was transmitted to the input of the multiplexor (DA). The three gate terminals on the JPI pin header were respectively linked to channels S11A, S13A, and S15A. The output signal of WEI could be directed through any of these three signals based on the selected output channel indicated by the signals from the WE_MUX_CHAN channels. These WE_MUX_CHAN_0-WE_MUX_CHAN_3 channels were connected to A0-A3 on the multiplexor, enabling the channel selection for the output.

Three pins from socket receptors connecting to CON2 and CON3 (CEO and RE0) were shorted together and connected to the REI and CEI channels. This combined signal was then directed to the three source channels on the JP1 pin header. Finally, three pins from CON4 (WE1) were linked to the drain terminal on JP1. Ultimately, the 45-degree pin header, JP1, was connected to a ribbon cable that runs to the previously mentioned deflection pin adapter board. Both boards were designed to fit inside the original 7.5×7.5 cm footprint of the MUX16 board so as not to increase the overall size of the housing.

### Connection Pin Force v Displacement

A study was conducted to determine a Force vs Displacement curve for the electrical connection pins of the disclosed testing apparatus. Determination of this force vs displacement relationship provides information regarding the limits of the force added to the pins such that they will be compressed enough to provide an electrical connection with the transducer without breaking. This relationship between force vs displacement is also important as both the pins and the gasket will be compressed, and both elements must be oriented and compressed together.

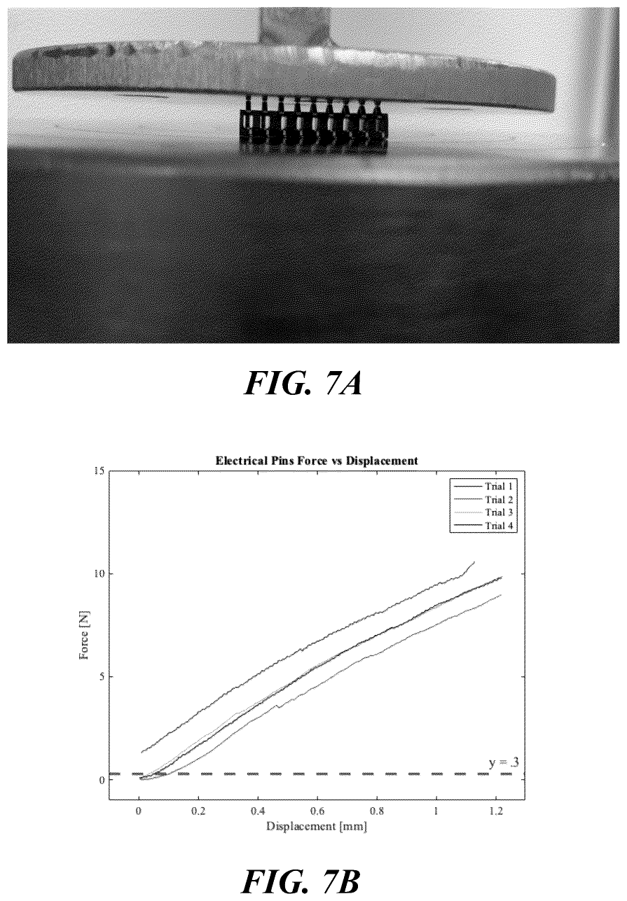

FIG. 7A shows the connection pin test setup, including the connection pins placed within a compression testing block on a universal testing machine (UTM). Software communicating with the UTM measured applied force, displacement, and time.

The procedure for the test is as follows: (1) add machine supports and compression measurement device; (2) connect the UTM to the software; (3) place connection pins on lower compression block and lower the top block until contact is just about to be made; (4) start the compression process and stop at 1.2 mm displacement to ensure the pins are not compressed enough to break—collecting data along the way; (5) remove the top compression block; and (6) save the data for analysis.

Four trials of the experiment were run to ensure an accurate relationship between the force applied to the pins and the distance that they compress. FIG. 7B shows a plot of the four trials showing the curves for force vs displacement. The average slope of these lines is 8.3 which means an applied force of 8.3 N will result in a displacement of 1 mm. A minimum of 0.3 N is required for electrical contact of the pins (dashed line), although a higher force will ensure that the pins are operating correctly. Therefore, a goal displacement of approximately 0.5 mm was selected for prototypical designs, resulting in a force of around 4 Newtons.

Gasket Seal Force. A study was conducted to find the applied force required to seal the gasket insert and prevent fluid from draining into the well. An adequate sealing force ensures that fluid is being sealed in order to create steady measurements, and it prevents fluid from reaching other parts of the housing that contain electronic components. This relationship between force vs. displacement of the gasket also helps to determine the exact location of the gasket, as the pins are being compressed with the gasket, and to orient the gasket such that both are operating correctly.

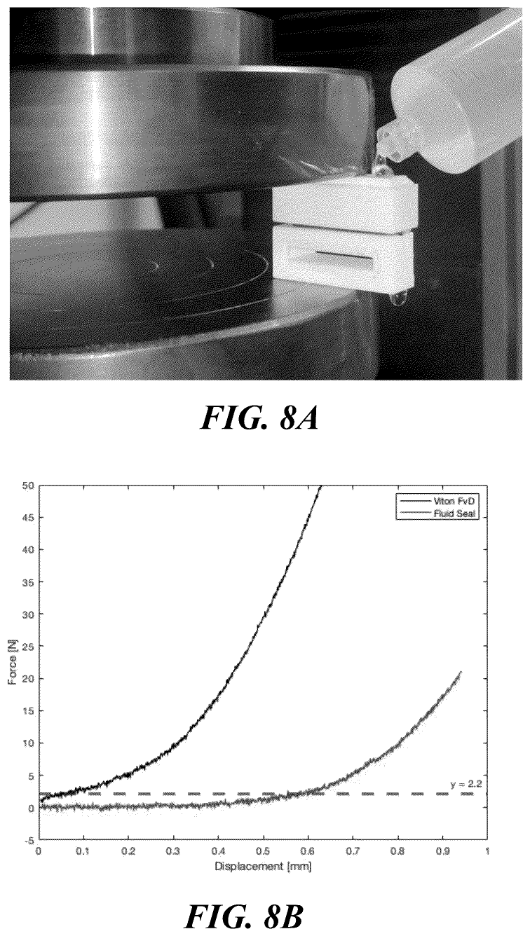

FIG. 8A shows the compression testing blocks used on a universal testing machine (UTM) with the gasket testing device therebetween. The bottom piece with the gasket and the top piece were 3D-printed parts simulating the top and bottom sections of the testing apparatus. Software communicating with the UTM measured applied force, displacement, and time.

The procedure for the test is as follows: (1) add machine supports and compression measurement device; (2) connect the UTM to the software; (3) insert the gasket into the bottom of the gasket testing device, keeping one edge accessible to fill the well with fluid; (4) lower the top block to just above the tested piece; (5) start the compression process; (6) continuously fill the well with fluid, as it drains with low compression; (7) stop a timer once the fluid is no longer draining from the well, collecting force data along the way; (8) stop the compression process and raise the top block; and (9) save the collected data for analysis.

A curve of force vs displacement for the gasket sealing test is shown in FIG. 8B. This test was run first to get a curve for the force vs displacement of the gasket material itself (black, upper curve). The red, lower curve shows data for when fluid was applied to the well, and the dashed line is the force at which the well sealed the fluid. Both curves represent the force vs. displacement of the gasket material, but the red, lower curve was run at a slower compression rate, and fluid was added throughout the test in order to calculate the force required to seal the well and prevent fluid from leaking. The dashed line is the force (˜2.2 N) at which the fluid was scaled. This value was found via a live video where the time was recorded when fluid in the well was no longer leaking. The data collected included time, and the corresponding force at the specific time was used.

Additional testing and experiments were performed that are not repeated in detail in this disclosure. For example, cyclic testing was performed on the testing apparatus and its associated components. In one study, the pin contacts were found to survive at least 11,000 cycles of compression when compressed not greater than 1 mm. In another study, the gasket was found to survive at least 4000 cycles of compression.

### Configuration of Certain Implementations

The construction and arrangement of the systems and methods, as shown in the various implementations, are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products, including machine-readable media for carrying or having machine-executable instructions or data structures stored thereon. Such machine-readable media can be any available media that can be accessed by a computer or special-purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a computer or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a computer, special-purpose computer, or special-purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A testing apparatus comprising:
a first section defining a recess configured to receive and align a transducer and comprising an alignment protrusion extending from a first surface of the first section;
a second section hinge-ably coupled to the first section and defining (i) a sample port extending therethrough from a first surface of the second section to a second surface of the second section, and (ii) an alignment opening extending from the second surface of the second section towards the first surface of the second section, the alignment opening configured to accept the alignment protrusion;
a gasket insert disposed within the sample port of the second section and defining at least one well configured to receive a fluid therein, wherein the at least one well is configured to align with a first portion of the transducer when placed in the recess of the first section; and
a connector coupled to the second surface of the second section, the connector having a plurality of flexible or spring-loaded contact pins extending away from the second surface of the second section, the plurality of contact pins being alignable with a second portion of the transducer when placed in the recess of the first section, the second portion having electrodes contact spaced apart from, and electrically connected to, the first portion of the transducer,
wherein the testing apparatus is movable between (i) an open configuration wherein the first surface of the first surface is spaced apart from and disposed at an angle with respect to the second surface of the second section and (ii) a closed configuration wherein the alignment protrusion of the first section is disposed within the alignment opening of the second section.

2. The testing apparatus of claim 1, wherein, when the testing apparatus is in the closed configuration, the at least one well is configured to receive a testing fluid and the gasket insert forms a fluid-tight seal with the first portion of the transducer.

3. The testing apparatus of claim 2, further comprising a printed circuit board in electrical communication with the plurality of contact pins, the printed circuit board configured to selectively apply or measure an electrical signal at each of the plurality of contact pins.

4. The testing apparatus of claim 3, wherein the printed circuit board is configured to interrogate the contact pins by application of a voltage to a first electrode of the second portion transducer.

5. The testing apparatus of claim 4, wherein the printed circuit board is configured to interrogate the contact pins to measure a concentration of at least one target element selected from the group consisting of potassium, nitrogen, and phosphate.

6. The testing apparatus of claim 3, wherein the printed circuit board is electrically coupled to the plurality of contact pins via a conductor routed through a hinge connecting the first section to the second section.

7. The testing apparatus of claim 1, wherein the transducer comprises a potentiostat electrode, amperostat electrode, coulometry electrode, or voltammetry electrode.

8. The testing apparatus of claim 1, wherein the first section further defines an overflow well surrounding the recess configured to receive the transducer, wherein the overflow well collects and redirects excess fluid towards an overflow container in fluid communication with the overflow well.

9. The testing apparatus of claim 1, wherein the at least one well of the gasket insert comprises three wells.

10. The testing apparatus of claim 1, wherein the alignment protrusion comprises a tapered protrusion and the alignment opening comprises a tapered recess matching that of the alignment protrusion, wherein the tapered protrusion and tapered recess are configured to urge the transducer to an aligned position in the recess while the flattening the transducer as the testing apparatus moves from the open configuration to the closed configuration.

11. The testing apparatus of claim 1, wherein one of the first section or the second section comprises a latch and the other one of the first section or the second section comprises an latch receiver dimensioned to accept the latch, wherein in the closed configuration the latch maintains the testing apparatus in the closed configuration.

12. The testing apparatus of claim 1, wherein the first section is configured to house a printed circuit board.

13. The testing apparatus of claim 1, wherein the plurality of contact pins are biased away from the second surface of the second section such that, in the closed configuration, the plurality of contact pins provide a spring force against the second portion of the transducer.

14. The testing apparatus of claim 1, wherein the recess of the first section further comprises a notch configured to align and orient the transducer and a divot configured to facilitate removal of the transducer.

15. A method of testing a sample fluid using the testing apparatus of claim 1.

16. The method of claim 15, wherein the sample fluid comprises a medical sample.

17. The method of claim 15, wherein the sample fluid comprises an agricultural or hydroponic sample.

18. The method of claim 15, wherein the sample fluid comprises an environmental sample.

19. The method of claim 15, wherein the sample fluid comprises a chemical manufacturing sample.
